(19) United States
(12) Patent Application Publication
(10) Pub. No.: US 2017/0296530 A1
(43) Pub. Date: Oct. 19, 2017

(54) Compositions and Methods for Treating and Inhibiting Viral Infections

(71) Applicant: Justice E. Obi, Saint Albans, NY (US)

(72) Inventor: Justice E. Obi, Saint Albans, NY (US)

(21) Appl. No.: 15/489,097

(22) Filed: Apr. 17, 2017

Related U.S. Application Data
(63) Continuation-in-part of application No. 13/932,445, filed on Jul. 1, 2013, now abandoned.
Application No. 13/932,445 is a continuation-in-part of application No. 13/687,273, filed on Nov. 28, 2012, now abandoned.
Application No. 13/687,273 is a continuation-in-part of application No. 13/540,749, filed on Jul. 3, 2012, now abandoned.
(60) Provisional application No. 61/825,569, filed on May 21, 2013.

(51) Int. Cl. A61K 31/4706; A61K 31/4164; A61K 9/00

(52) U.S. Cl. CPC A61K 31/4706; A61K 9/0014; A61K 31/4164

(57) ABSTRACT

Compositions and methods for the treatment, as well as the inhibition and prevention, of an infection of the papillomavirus and the epithelial lesions, namely, the warts of the skin and mucosal surfaces, associated therewith, in a mammalian host, as well as methods of inhibiting the replication of a papillomavirus in an infected cell, are provided. The compositions comprise a therapeutically effective amount of an active ingredient comprising at least one compound selected from the group consisting of chloroquine, hydroxychloroquine, amodiaquine, or in each case, a pharmaceutically acceptable salt thereof. The methods comprise topically administering a therapeutically and/or antivirally effective amount of such a compound to a mammalian host, such as a human being, in need of such treatment, although alternatively other routes of administration may be used, including but not limited to transdermal, transmucosal, respiratory, and by injection. The compositions optionally also comprise one or more pharmaceutically acceptable non-active ingredients.

### REFERENCE TO RELATED APPLICATIONS

This application is a continuation-in-part of and claims the benefit of non-provisional application U.S. Ser. No. 13/932,445, filed Jul. 1, 2013 entitled Compositions And Methods For Treating And Inhibiting Viral Infections which claims the benefit of application Ser. No. 13/687,273, filed Nov. 28, 2012, which was a continuation-in-part of prior co-pending application Ser. No. 13/540,749, filed Jul. 3, 2012, This application also claims the benefit under 35 U.S.C. .sctn.119(e) of prior U.S. Provisional Patent Application Ser. No. 61/825,569, filed May 21, 2013. All of said related applications are incorporated herein in their entireties.

### TECHNICAL FIELD

This invention relates, in general, to compositions and methods for treating and inhibiting papillomavirus infections and removing warts of papillomavirus genesis in mammalian hosts, and in particular, to the treatment and prevention of genital warts and other warts and lesions of the skin and mucosal surfaces that are associated with infections of the papillomavirus in mammals. More specifically, this invention relates to compositions and methods for treating and inhibiting replication of the human papillomarivus, thereby eliminating the cause of such warts and lesions.

### BACKGROUND

The human papillomavirus is a virus of the papillomavirus family that infects the epidermis of human beings. Such infections may result in epithelial lesions or growths, particularly warts, also known as condylomas or papillomas. Many different human papillomavirus types have been identified, which are usually transmitted through skin-to-skin contact (or mucous membrane contact), including sexual contact. Human papillomavirus types 6 and 11 are most often associated with unsightly and embarrassing, but usually non-cancerous, warts on the male genitalia, in and around the vagina, in and around the anus, in and around the throat, including the larynx, or other skin and/or mucous membrane areas. Other human papillomavirus types include, but are not limited to, types 16 and 18, which are also transmitted through skin-to-skin contact (or mucous membrane contact), including sexual contact, and are associated with unsightly and often embarrassing anogenital warts. The human papillomavirus is also known generally to play a role in and may in fact lead to, several types of cancers, including cervical, vaginal, vulvar, penile, anal, rectal and/or oropharyngeal cancers, although types 16 and 18 are associated with a higher risk of cancer than types 6 and 11 and other types. Other human papillomavirus types include type 1, which is associated with unsightly but usually non-cancerous warts that appear on the feet. The present invention may be used to treat and inhibit infections caused by all of the aforementioned human papillomavirus types, but the invention is not limited to the treatment and inhibition of infections caused solely by the human papillomavirus types mentioned above.

Infections caused by the human papillomavirus (“HPV”), and warts associated with that virus, have often been treated and/or removed using one of the following compositions and/or methods: cryotherapy, which involves freezing the abnormal cells with liquid nitrogen; conization, or a cone biopsy, which removes the abnormal areas surgically; or Loop Electrosurgical Excision Procedure (LEEP), where the abnormal cells are removed with an electrical current. For warts in less sensitive areas, acids may be applied to assist in terminating the wart; canadid antigen may be injected to stimulate the immune system to fight off the wart; a topical medication called imiquimod may be applied; or no treatment at all may be used, which implies simply waiting to see if the cells can heal on their own.

However, many of these compositions and/or methods include burning or freezing away the wart (using chemicals or an electrical current), which can often be a painful process, and in any event can leave unsightly scarring. Others utilize a medication that modifies the immune response to the virus in order to treat the wart, but these often require more time to fight the virus and terminate the wart, which does not always happen. Additionally, while these methods may be used to target and treat existing warts, they do not necessarily treat the underlying virus which may result in a recurrence of the warts, as the warts are a manifestation of the viral infection.

Various patents and other prior art have been proposed for treating warts and related conditions by infusing or injecting one or more pharmaceuticals into a lesion, i.e. systemic treatments. See Wilson (“Clinical Evaluation of Intralesional Administration . . . ”), Baldwin et al. (U.S. Pat. No. 5,021,426), and Albahri (U.S. Patent App. No. 2004/0109899). More particularly, Wilson does not discloses systemic treatment using chloroquine and amodiaquine but not topical administration of the same or in the dosages or administration conditions that will be described later. It has been incorrectly assumed or concluded by some that (1) since systemic treatment, such as through needle injections of anti-malarial agents like chloroquine, have shown medical efficacy (e.g. Wilson), and (2) since topical treatments are sometimes (with some drugs) similarly effective to treat the same condition as treated by injection or systemic treatment (e.g. Albahri), that topical treatment of HPV and other wart conditions would also work against HPV lesions and, as a result, be merely obvious to one of ordinary skill in the art.

But, such a conclusion is erroneous with respect to treating human papillomavirus in humans patients. A scholarly article in 2014 entitled “Complete Clearance of Cutaneous Warts with Hydrochloroquine: Antiviral Action?” confirms the knowledge in the art at the time of the present invention. More particularly, it was observed that systemic treatments (which was prescribed and shown to be very effective in the case studied by the article), including use of the chloroquine family, had shown high effectiveness and are commonly prescribed due to the noted historical limitations of topical therapy for warts. Therefore, a topical treatment that results in complete eradication of HPV lesions would be unexpected indeed based on documented prior art.

### SUMMARY OF THE INVENTION

It is therefore one of the primary objects of the present invention to provide compositions and methods for topically treating infections caused by the human papillornavirus and for removing warts associated with that virus, particularly, but not limited to, those associated with types 1, 6, 11, 16 and 18.

Another object of the present invention is to provide compositions and methods for treating infections caused by the human papillomavirus and for removing warts associated with that virus in which the compositions are safe enough to be used topically on highly sensitive regions of the skin, such as may be found in the genital areas.

Still another object of the present invention is to provide compositions and methods for treating infections caused by the human papillomavirus and for removing warts associated with that virus which can be administered to humans through different routes of administration and in a variety of forms.

Yet another object of the present invention is to provide compositions and methods for treating infections caused by the human papillomavirus and for removing warts associated with that virus which can also be used to cover scars left after conventional surgical removal of such warts, in order to prevent re-growth of such warts.

Still one other object of the present invention is to provide compositions and methods for treating infections caused by the human papillomavirus and for removing warts associated with that virus which provide quick relief and termination of the wart, as well as sustained relief to prevent additional warts from forming, by inhibiting replication of virus.

The present invention provides compositions and methods for treating an infection associated with the human papillomavirus: such an infection may be manifested in a human as epithelial lesions, including warts, and particularly skin warts, lesions and anogenital warts. The compositions of the invention for treating and removing such warts comprise at least one compound selected from the group consisting of chloroquine, hydroxychloroquine and amodiaquine. More particularly, the compositions comprise either chloroquine alone or hydroxychloroquine alone or amodiaquine alone, or pharmaceutically acceptable salts thereof, as the active ingredient, or combinations of any two, or all three, of those compounds, or pharmaceutically acceptable salts thereof, as the active ingredients. Optionally, the compositions may also comprise suitable pharmaceutically acceptable carriers, excipients and/or adjuvants, and/or other non-active ingredients, such as ethyl alcohol, lidocaine, epinephrine and/or diphenhydramine. It is believed that the compositions of the invention act by inhibiting replication of human papillorravirus which causes such warts.

The methods of the invention comprise administering a pharmaceutical composition comprising at least one compound selected from the group consisting of chloroquine, hydroxychloroquine, amodiaquine and pharmaceutically acceptable salts thereof, to a human being, using one or more routes of administration that are well known in the art. At present, the following are the preferred routes of administration: topical (epicutaneous), in forms such as a gel, cream, spray, soap or other bathing apparatus; transdermal, in the form of a patch; transmucosal (also known as pharmaceutical pessary delivery) through the vagina or rectum, in the form, for example, of an ovule or suppository, respectively; nasal, such as delivery by inhalation of aerosol droplets produced, for example, with the aid of a nebulizer; and infusion under the skin in the form of an injection; Other routes for administering pharmaceutical compositions according to the invention are possible.

Thus, one aspect of the present invention generally concerns methods for the treatment of a human or mammalian subject suffering from an existing papillomavirus infection by inhibiting further replication of the virus. In one embodiment of this aspect, the methods comprise administering a therapeutically effective amount of chloroquine or a pharmaceutically acceptable salt thereof to the subject. In another embodiment the methods comprise administering a therapeutically effective amount of hydroxychloroquine or a pharmaceutically acceptable salt thereof to the subject. In yet another embodiment the methods comprise administering a therapeutically effective amount of amodiaquine or a pharmaceutically acceptable salt thereof to the subject. In still another embodiment of this aspect of the invention, the methods comprise administering a therapeutically effective amount of chloroquine or a pharmaceutically acceptable salt thereof together with a therapeutically effective amount of hydroxychloroquine or a pharmaceutically acceptable salt thereof to the subject. In yet another embodiment, the methods comprise administering a therapeutically effective amount of chloroquine or a pharmaceutically acceptable salt thereof together with a therapeutically effective amount of amodiaquine or a pharmaceutically acceptable salt thereof to the subject. In still another embodiment, the methods comprise administering a therapeutically effective amount of hydroxychloroquine or a pharmaceutically acceptable salt thereof together with a therapeutically effective amount of amodiaquine or a pharmaceutically acceptable salt thereof to the subject. In yet another embodiment, the methods comprise administering a therapeutically effective amount of chloroquine or a pharmaceutically acceptable salt thereof together with a therapeutically effective amount of hydroxychloroquine or a pharmaceutically acceptable salt thereof and a therapeutically effective amount of amodiaquine or a pharmaceutically acceptable salt thereof to the subject.

Another aspect of the invention generally concerns methods for the prevention or inhibition of the development or recurrence of the development in a human or mammalian subject of an infection of the human papillomavirus by inhibiting replication of the virus. In one embodiment of this aspect, the methods comprise administering an antivirally effective amount of chloroquine or a pharmaceutically acceptable salt thereof to the subject. In another embodiment the methods comprise administering an antivirally effective amount of hydroxychloroquine or a pharmaceutically acceptable salt thereof to the subject. In still anther embodiment the methods comprise administering an antivirally effective amount of amodiaquine or a pharmaceutically acceptable salt thereof to the subject. In still another embodiment of this aspect of the invention, the methods comprise administering an antivirally effective amount of chloroquine or a pharmaceutically acceptable salt thereof together with an antivirally effective amount of hydroxychloroquine or a pharmaceutically acceptable salt thereof to the subject. In yet another embodiment, the methods comprise administering an antivirally effective amount of chloroquine or a pharmaceutically acceptable salt thereof together with an anitvirally effective amount of amodiaquine or a pharmaceutically acceptable salt thereof to the subject. In still another embodiment, the methods comprise administering an antivirally effective amount of hydroxychloroquine or a pharmaceutically acceptable salt thereof together with an antivirally effective amount of amodiaquine or a pharmaceutically acceptable salt thereof to the subject. In yet another embodiment, the methods comprise administering an antivirally effective amount of chloroquine or a pharmaceutically acceptable salt thereof together with an antivirally effective amount of hydroxychloroquine or a pharmaceutically acceptable salt thereof and an anitvirally effective amount of amodiaquine or a pharmaceutically acceptable salt thereof to the subject.

Another aspect of the invention generally concerns pharmaceutical compositions for the treatment of a papillomavirus infection in a human or mammalian host by inhibiting replication of the virus. In one embodiment of this aspect, the compositions comprise a therapeutically effective amount of chloroquine or a pharmaceutically acceptable salt thereof. In another embodiment the compositions comprise a therapeutically effective amount of hydroxychloroquine or a pharmaceutically acceptable salt thereof. In still another embodiment the compositions comprise a therapeutically effective amount of amodiaquine or a pharmaceutically acceptable salt thereof. In still another embodiment of this aspect of the invention, the compositions comprise a therapeutically effective amount of chloroquine or a pharmaceutically acceptable salt thereof together with a therapeutically effective amount of hydroxychloroquine or a pharmaceutically acceptable salt thereof. In yet another embodiment, the compositions comprise a therapeutically effective amount of chloroquine or a pharmaceutically acceptable salt thereof together with a therapeutically effective amount of amodiaquine or a pharmaceutically acceptable salt thereof. In still another embodiment, the compositions comprise a therapeutically effective amount of hydroxychloroquine or a pharmaceutically acceptable salt thereof together with a therapeutically effective amount of amodiaquine or a pharmaceutically acceptable salt thereof. In yet another embodiment, the compositions comprise a therapeutically effective amount of chloroquine or a pharmaceutically acceptable salt thereof together with a therapeutically effective amount of hydroxychloroquine or a pharmaceutically acceptable salt thereof and a therapeutically effective amount of amodiaquine or a pharmaceutically acceptable salt thereof.

Another aspect of the invention generally concerns methods for the treatment of a papillomavirus infection in a cell of human or mammalian origin, as well as methods of inhibiting the replication of a papillornavirus in a cell of human or mammalian origin infected with such a virus. In one embodiment of this aspect, the methods comprise exposing the cell to an antivirally effective amount of chloroquine or a pharmaceutically acceptable salt thereof. In another embodiment the methods comprise exposing the cell to an antivirally effective amount of hydroxychloroquine or a pharmaceutically acceptable salt thereof. In yet another embodiment the methods comprise exposing the cell to an antivirally effective amount of amodiaquine or a pharmaceutically acceptable salt thereof. In still another embodiment of this aspect of the invention, the methods comprise exposing the cell to an antivirally effective amount of chloroquine or a pharmaceutically acceptable salt thereof together with an antivirally effective amount of hydroxychloroquine or a pharmaceutically acceptable salt thereof. In yet another embodiment, the methods comprise exposing the cell to an antivirally effective amount of chloroquine or a pharmaceutically acceptable salt thereof together with an antivirally effective amount of amodiaquine or a pharmaceutically acceptable salt thereof. In still another embodiment, the methods comprise exposing the cell to an antivirally effective amount of hydroxychloroquine or a pharmaceutically acceptable salt thereof together with an antivirally effective amount of amodiaquine or a pharmaceutically acceptable salt thereof. In yet another embodiment, the methods comprise exposing the cell to an antivirally effective amount of chloroquine or a pharmaceutically acceptable salt thereof together with an antivirally effective amount of hydroxychloroquine or a pharmaceutically acceptable salt thereof and an antivirally effective amount of amodiaquine or a pharmaceutically acceptable salt thereof.

These and other aspects, features, objects and advantages of the present invention will become more apparent to those skilled in the art from the following detailed description of the presently most preferred embodiments thereof.

In a further aspect, the current disclosure provides methods of using compositions comprising metronidazole for the treatment of viral infections.

### DETAILED DESCRIPTION OF THE PREFERRED EMBODIMENT

The disclosure provides compositions that comprise one or more active ingredients from the group consisting of chloroquine, amodiaquine, and an antibiotic. According to some embodiments, the antibiotic is metronidazole.

The present invention resides in the discovery that the known compounds chloroquine, hydroxychloroquine and amodiaquine, or pharmaceutically acceptable salts thereof, all of which have been used previously as antimalarial agents and/or to treat disorders of the immune system, also have utility, when applied topically as described below, in treating infections of the human and other mammalian papillomaviruses, and in particular, in treating and in inhibiting replication of such viruses and in removing the warts associated with such infections, as well as in preventing the recurrence of such warts.

The molecular structures of chloroquine (IUPAC name: (RS)—N′-(7-chloroquinolin-4-yl)-N,N-diethyl-pentane-1,4-diamine), hydroxychloroquine (IUPAC name: (RS)-2-[{4-[(7-chloroquinolin-4-yl)amino]pentyl}(ethyl)amino]ethanol) and amodiaquine (IUPAC name: 4-[(7-chloroquinolin-4-yl)amino]-2-[(diethylamino)methyl]phenol) are provided below, as Formula I, Formula II, and Formula III, respectively.

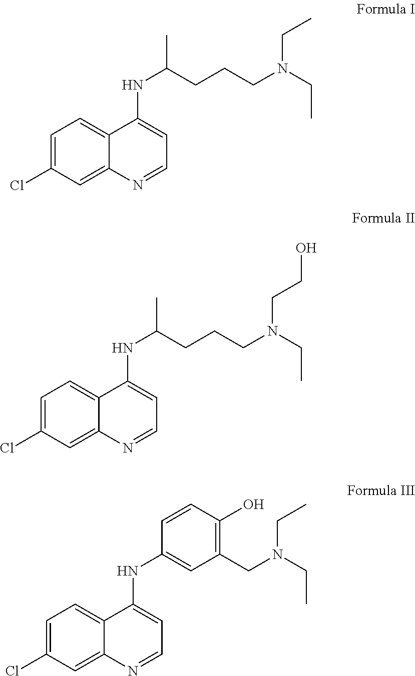 [SMILES: CCN(CC)CCCC(C)Nc1ccnc2cc(Cl)ccc12 ; CCN(CCO)CCCC(C)Nc1ccnc2cc(Cl)ccc12 ; CCN(CC)Cc1cc(Nc2ccnc3cc(Cl)ccc23)ccc1O]

As is known, and as can be seen in the structural formulas set out above, these three compounds are related to one another in that their structures have a heterodicyclic portion in common, namely, the 7-chloro derivative of the 4-aminoquinoline moiety. Chloroquine (Formula I) has been marketed under the brand name RESOCHIN, while hydroxychloroquine (Formula II) has been marketed under the brand name PLAQUENIL, and amodiaquine (Formula III) has been marketed under the brand name CAMOQUIN. Chloroquine is commercially available as the pharmaceutically acceptable salt chloroquine phosphate, and may be purchased in solid (tablet) form from a wide variety of sources such as Ohm Laboratories, Inc of North Brunswick, N.J., U.S.A., while a ready-made aqueous solution may be purchased from a wide variety of sources, including Sal Parenterals (P) Ltd., of Hyderabad, India, or Scott Edil Pharmacia Ltd., of Jhannajri, India. Hydroxychloroquine, in solid (tablet) form, is commercially available as the pharmaceutically acceptable salt hydroxychloroquine sulfate, and may be purchased from a wide variety of sources such as West-Ward Pharmaceutical Corporation of Eatontown, N.J., U.S.A. Amodiaquine, in solid (tablet) form, is commercially available as the pharmaceutically acceptable salt amodiaquine hydrochloride, and may be purchased from a wide variety of sources, including Parke, Davis & Company, a division of Pfizer Inc., headquartered in New York, N.Y., U.S.A.

In certain embodiments, the compositions of the disclosure comprise an antibiotic. In some embodiments, the antibiotic is metronidazole. Metronidazole is a nitroimidazole antibiotic and antiprotozoal medication. It can be given via oral or parenteral (including topical, intravenous, and intravaginal) administration. Metronidazole has been marketed under the names METROGEL, FLAGYL, NORITATE, METROCREAM, ROSADAN, and METROLOTION, among others. The molecular structure of metronidazole (IUPAC name: 2-(2-methyl-5-nitro-1H-imidazol-1-yl)ethanol) is provided below, as Formula IV.

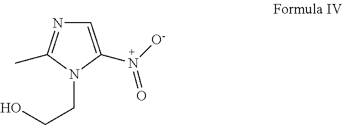

In certain embodiments, the compositions of the disclosure comprise either chloroquine (Formula I) alone or amodiaquine (Formula III) alone as the active ingredient, or chloroquine combined with amodiaquine, or chloroquine combined with metronidazole (Formula IV), or amodiaquine combined with metronidazole, or chloroquine combined with both amodiaquine and metronidazole, as the active ingredients.

In general, as mentioned above and as set forth in further detail below, the methods of the disclosure comprise, in certain embodiments, administering the pharmaceutical compositions of the disclosure comprising the active ingredient or ingredients to a subject such as a human being using one or more of the specific routes of administration, which include direct topical (epicutaneous) administration, in forms such as gel, douche, cream, lotion, solution, spray, soap or other bathing apparatus; transdermal administration, in the form of a patch; transmucosal administration (also known as pharmaceutical pessary delivery) through the vagina or rectum, in the form, for example, of an ovule or a suppository, including a vaginal suppository; respiratory administration, such as delivery via inhalation through the nostrils and nasal passages of aerosol droplets produced, for example, with the aid of a nebulizer; and infusion under the skin in the form of an intra-epidermal injection.

Although the compositions and methods of the disclosure will be illustratively described hereinafter with reference to topical, transmucosal, oral and other routes of administration, it should be understood that the disclosure is not limited to the specific cases described, but extends also to the use of other compatible routes for administering pharmaceutical compositions according to the disclosure, as will be evident to those skilled in the art, including but not limited to other topical and/or parenteral routes, such as buccal, conjunctival, endotracheal, intramuscular, intravascular, laryngeal, or ophthalmic, or even enteral (oral) routes, any one or more of which may be used. Suitable formulations for any route of administration that is ultimately selected are known and are described in well-known texts, including for example Remington, The Science and Practice of Pharmacy, 21st edition, 2005, Mack Publishing Company, Easton, Pa., and therefore such formulations may easily be prepared by those of ordinary skill in the art.

It is well understood, however, that proper dosages of any medication may vary from one individual to another, depending on many factors such as the intensity of the affliction and the selected route of administration, as well as the weight, age and gender of the subject or patient. Therefore, the effective dosages of the pharmaceutical compositions of the present disclosure should be determined by a specialist in this matter, such as a medical doctor or other health care provider, depending on these and other parameters. Nevertheless, for the sake of illustration only, exemplary formulations, preparation procedures and dosages, for topical, transmucosal, and other routes of administration, are provided herein for the sake of guidance.

When the compositions of the disclosure comprising one or more of chloroquine, amodiaquine and metronidazole as the only active ingredient(s) are to be administered topically (epicutaneously), in the form of a gel or a spray, the compositions may comprise chloroquine and/or amodiaquine and/or metronidazole plus optional non-active ingredients. The non-active ingredients may comprise sodium carboxymethylcellulose, a preservative (for example, methylparaben, for example at 0.5%), purified water, Versatile gel base, as well as ethyl alcohol and peppermint spirit oil, and the gel form may additionally comprise lidocaine jelly or ointment, while the spray form may additionally comprise lidocaine as a 2% solution. All of the non-active ingredients are conventional and are available commercially from a wide variety of sources. The non-active ingredients are exemplary only, but are included for the following purposes: the lidocaine functions as an analgesic, the ethyl alcohol functions as an antiseptic, while the peppermint spirit oil functions to provide a pleasing odor. The compositions of the disclosure comprising one or more of chloroquine, amodiaquine and metronidazole are also provided in suppository and douche forms. The suppository forms may comprise chloroquine and/or amodiaquine and/or metronidazole plus optional non-active ingredients. The douche forms may comprise chloroquine and/or amodiaquine and/or metronidazole plus optional non-active ingredients. The non-active ingredients in the suppository form may comprise Polybase (for example, containing PEG 400, PEG 8000 and Polysorbate 80), and citric acid, while the non-active ingredients in the douche form may comprise purified water for irrigation, sodium chloride (for example 0.9%, or normal saline), citric acid, a preservative (for example, sodium benzoate).

Particular embodiments of compositions for the gel, suppository and douche forms may be prepared as set forth in the Formulation Examples below.

The gel forms of these medications may be administered to a subject as follows: The affected areas are first cleaned with one or more alcohol swabs. Thereafter, an amount of the gel approximately equal to the surface area of each affected area (or an amount equal to the surface area of the tip of a finger) is applied to the affected area, after which that area may optionally be covered with a sterile bandage. The gel form may be administered to the subject in the foregoing manner once or twice a day, for approximately one to four weeks, until return of the affected area(s) to a normal skin appearance.

The spray forms of these medications may be administered to a subject in the same manner, although the dosage may be two puffs applied to the affected areas once or twice daily.

The suppository forms for vaginal administration of these compositions may be administered to a subject by placing the “bullet”, or suppository, into the vagina. The suppository form for vaginal administration may be administered to the subject once or twice a day, for approximately one to four weeks, until return of the affected area(s) to a normal skin appearance. The suppository form for vaginal administration may be administered before or at bedtime.

The douche forms for vaginal or oral administration of these compositions may be administered vaginally using a bottle or other suitable apparatus. It may also be administered orally, where needed, by gargling of the douche solution. Either form (as appropriate) may be administered to the subject once or twice a day, for approximately one to four weeks.

### A. TERMS, DEFINITIONS AND ABBREVIATIONS

As used herein and unless otherwise expressly noted or required by the context, all percentages refer to percentages by weight (wt-%) of the total composition (w/w).

As used herein in connection with a measured quantity, for example weight, “about” refers to that variation in the measured quantity as would be expected by one skilled in the art exercising a level of care commensurate with the objective of the measurement and the equipment used, and includes uncertainties that may be introduced by mathematical rounding errors.

As used herein, the term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile may be used. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### B. EMBODIMENTS

The following examples are provided to further elucidate the advantages and features of the present application, but are not intended to limit the scope of the application. The examples are for the illustrative purposes only. USP pharmaceutical grade products were used in preparing the formulations described below.

### Formulation Examples

The formulation percentage range covers from 2.5% to 25% of the

The present invention resides in the discovery that the known compounds chloroquine, hydroxychloroquine and amodiaquine, or pharmaceutically acceptable salts thereof, all of which have been used previously as antimalarial agents and/or to treat disorders of the immune system, also have utility in treating infections of the human and other mammalian papillomaviruses, and in particular, in treating and in inhibiting replication of such viruses and in removing the warts associated with such infections, as well as in preventing the recurrence of such warts.

The molecular structures of chloroquine, hydroxychloroquine and amodiaquine are provided below, as Formula (I), Formula (II) and Formula (III), respectively. active ingredient, whether it is one active ingredient or two active ingredients, or a mixture of three active ingredients.

For topical treatment of the papillomavirus family of viruses that infects the epidermis of human beings disease:

### Gel Formula 1

For Chloroquine compounding, to make 10% gel tube of 60 grams:

| | | |
|---|---|---|
| 1) Chloroquine phosphate pure powder 10% equals to | 7 | grams |
| 2) Sodium Carboxymethylcellulose is | 1.2 | grams |
| 3) Methylparaben (0.5%) (as preservative) is | 1.2 | grams |
| 4) Purified water is | 10 | ml |
| 5) Versatile gel base is | 41 | grams (ml) |

Sufficient to make 60 gram tube gel of Chloroquine 10%. To be applied about 2 times a day for about 14 days to cover the affected area topically. The composition may be prepared by crushing the chloroquine phosphate tablets in a mortar or using chloroquine phosphate powder, and then adding each of the non-active ingredients, in the sequence listed above, sufficient to make a total of approximately 60 gm of the gel. The gel form may then be transferred to a tube or other appropriate container.

### Gel Formula 2

For Chloroquine compounding, to make 10% gel of three compounds of 60 grams:

| | | |
|---|---|---|
| 1) Chloroquine | 2.2 | grams |
| 2) Amodiaquine | 2.2 | grams |
| 3) Metronidazole | 2.2 | grams |
| 2) Sodium Carboxymethylcellulose is | 1.2 | grams |
| 3) Methylparaben (0.5%) (as preservative) is | 1.2 | grams |
| 4) Purified water is | 10 | ml |
| 5) Versatile gel base is | 41 | grams (ml) |

Sufficient to make 60 gram tube gel. To be applied about 2 times a day for about 14 days to cover the affected area topically. The composition may be prepared by adding the chloroquine, amodiaquine and metronidazole, and then adding each of the non-active ingredients, in the sequence listed above, sufficient to make a total of approximately 60 gm of the gel. The gel form may then be transferred to a tube or other appropriate container.

### Vaginal Suppository Formula 1

To formulate 24 bullet-shaped suppositories to treat the viral infections according to the present invention.

| 1) Chloroquine | 6 | grams of pure powder |
|---|---|---|
| 2) Polybase containing | PEG 400, PEG 8000 and Polysorbate 80 | |
| 3) Citric acid powder | 1.5 | grams |

Sufficient to make 24 bullet shaped vaginal suppositories. To be applied vaginally at bedtime for about 14 days. Optional active ingredient: Metronidazole—6 gram/60 ml in which each ml contains 100 mg of Metronidazole. The vaginal suppository composition may be prepared by adding the chloroquine (and metronidazole if included), and then adding each of the non-active ingredients, in the sequence listed above, sufficient to make the suppository.

### Vaginal Suppository Formula 2

To formulate 24 bullet-shaped suppositories to treat to treat the viral infections according to the present invention.

| 1) Amodiaquine | 7 | grams of pure powder |
|---|---|---|
| 2) Metronidazole | 6 | grams |
| 2) Polybase containing | PEG 400, PEG 8000 and Polysorbate 80 | |
| 3) Citric acid powder | 1.5 | grams |

Sufficient to make 24 bullet shaped vaginal suppositories. To be applied vaginally at bedtime for about 14 days. The vaginal suppository composition may be prepared by adding the amodiaquine and metronidazole, and then adding each of the non-active ingredients, in the sequence listed above, sufficient to make the suppository.

### Douche Solution

To make 10% Chloroquine douche in 4.5 oz (133 ml). To be applied vaginally or orally (for gargling) at bedtime for about 14 days.

| | | |
|---|---|---|
| 1) Chloroquine | 15 | gm pure powder |
| 2) Purified water for irrigation | 100 | ml |
| 3) Purified Sodium Chloride 0.9% | 15 | ml |
| (normal saline) | | |
| 4) Citric acid | 2 | ml |
| 5) Sodium benzoate (as preservative) | 1 | ml |

Optional active ingredient: Metronidazole—6 gram/60 ml in which each ml contains 100 mg of Metronidazole. The douche composition may be prepared by adding the chloroquine (and metronidazole if included), and then adding each of the non-active ingredients, in the sequence listed above, sufficient to make the suppository.

### Treatment Examples

In vivo experimental results which demonstrate the efficacy of the foregoing compositions are set forth below. Specifically, the following working examples illustrate both the manner in which a representative sample of the compositions of the present disclosure have been used in human subjects suffering from at least symptom associated with the viral conditions according to the present invention.

### Example 1

A juvenile male, specifically a 12-year old boy of Hispanic ancestry, was observed as having warts on three out of five fingers of the right hand. These warts were first filed in the manner set forth above for better contact with the medication, and they were then treated with a composition in gel form containing hydroxychloroquine as the only active ingredient (prepared in the manner set forth above for such compositions). This gel composition was applied to each of the warts, in an amount approximately equal to the surface area of each wart, once or twice a day for approximately one week, following which it was observed that all of the warts had disappeared completely, without leaving any visible scarring.

### Example 2

Another juvenile male, specifically a 15-year old boy also of Hispanic ancestry, presented with warts on his fingers. The same composition as in Example 1 was applied, in the same manner and with the same frequency as in Example 1, and after approximately one week of such treatments, similar results were observed, that is. all of the warts had disappeared completely, without leaving any visible scarring.

### Example 3

An adult male, specifically a 55-year old man of Hispanic ancestry, was observed to have genital warts, specifically, warts scattered about the skin of the scrotum. The same composition as in Example 1 was applied, in the same manner and with the same frequency as in Example 1, and after approximately one week of such treatments it was observed that 50% of the mass of the warts had disappeared. Thereafter, the patient switched to treatments with a composition in gel form containing chloroquine as the only active ingredient (prepared in the manner set forth above for such compositions), and after two days of similar twice-daily treatments with the latter composition, it was observed that the remaining 50% of the mass of the warts had disappeared.

### Example 4

An adult female, specifically a 50-year old black woman, and her male partner, a 24-year old man of mixed Hispanic and black ancestry, both of whom presented with genital warts. The female, who was also living with a human immunodeficiency virus (HIV) infection, reported warts on her labia majora, which were then treated with a composition in gel form containing chloroquine as the only active ingredient (prepared in the manner set forth above for such compositions). This gel composition was applied to each of the warts, in an amount approximately equal to the surface area of each wart, once or twice a day for approximately four weeks, after which the patient reported that she observed a great deal of improvement. Thereafter, the female patient switched to treatments with a composition in gel form containing chloroquine and amodiaquine as the active ingredients (prepared in the manner set forth above for such compositions), and after approximately two weeks of similar twice-daily treatments with the latter composition, she reported that the warts had disappeared.

The same treatment regime was used on her male partner, who presented with warts on his penis, and who, in addition, reported having been born with HIV. After three months it was observed that the warts had disappeared completely. It is believed that this patient may have required a longer duration of treatment to achieve a successful result due to his underlying HIV infection, which possibly may have reduced the ability of his immune system to combat the human papillomavirus infection.

### Example 5

Another adult male, specifically a 36-year old man of Hispanic ancestry, was observed as having warts on one of his fingers. The same composition as in Example 1 was applied, in the same manner and with the same frequency as in Example 1, and after two days of such treatments, similar results were observed; that is, all of the warts had disappeared completely, without any visible scarring.

### Example 6

Another adult female, specifically a 45-year old woman of Hispanic ancestry, presented with warts on her fingers. These warts were treated with a composition in gel form containing chloroquine as the only active ingredient (prepared in the manner set forth above for such compositions), which was applied in the same manner as in Example 1. After three days of such treatments, the patient reported that all of the warts had disappeared completely, without any visible scarring.

### Example 7

Another juvenile male, specifically a 16-year old boy also of Hispanic ancestry, presented with two warts on his hand. After filing, a composition in gel form containing amodiaquine as the only active ingredient (prepared in the manner set forth above for such compositions) was applied to each wart, in the same manner and with the same frequency as in Example 1, and after approximately one week of such treatments, the warts were reduced in size by approximately 50%. Following a second week of similar treatments, both of the warts had disappeared completely.

### Example 8

Two juvenile females, specifically a 16-year old girl and her 14-year old sister, both also of Hispanic ancestry, each presented with warts on their feet—the 16-year old had two large warts on one foot, which made walking difficult for her without pain, while the 14-year old had one smaller wart on one of her feet. After filing, a composition in gel form containing the combination of chloroquine and hydroxychloroquine as the active ingredients (prepared in the manner set forth above for such compositions) was applied generously to all of the warts on the feet of both patients. After approximately three weeks of such treatments twice a day, all of the warts had disappeared completely from the feet of both patients.

### Example 9

An adult male, specifically a 50-year old West African man from Senegal, presented with many different warts on his feet. After filing, a composition in gel form containing the combination of chloroquine, hydroxychloroquine and amodiaquine as the active ingredients (prepared in the manner set forth above for such compositions) was applied to the warts, as well as to the areas of his feet that were unaffected. After two weeks of such treatments twice a day, all of the warts had disappeared completely from both of his feet, allowing the patient to walk and wear shoes comfortably.

### Example 10

An adult male, specifically a 50-year old West African man from Nigeria, was observed to have two (2) genital warts on the shaft of his penis. These warts were treated with a composition in gel form containing chloroquine as the only active ingredient (prepared in the manner set forth above for such compositions). This gel composition was applied to each of the warts, in an amount approximately equal to the surface area of each wart, once a day for two weeks, following which it was observed that both warts had disappeared completely, leaving the skin flat. This patient was observed as never having a recurrence of the warts.

### Example 11

An adult male, specifically a 40-year old West African man from Nigeria, was observed to have a genital wart on the shaft of his penis. This wart was treated with a composition in gel form containing chloroqine and amodiaquine as the active ingredients (prepared in the manner set forth above for such compositions). This gel composition was applied to the wart, in an amount approximately equal to the surface area of the wart, and the area was then covered with an adhesive bandage, once a day for two weeks, following which it was observed that the wart had disappeared completely, leaving the skin flat. This patient was observed as never having a recurrence of the warts.

### Example 12

A juvenile female, specifically a 15-year old girl of Nigerian ancestry, was observed as having two plantar warts (located on the bottom of her right foot). These warts were treated with a composition in gel form containing chlorogine and hydroxychloroquine as the active ingredients (prepared in the manner set forth above for such compositions). This gel composition was applied to each of the warts, in an amount approximately equal to the surface area of each wart, and the area was then covered with an adhesive bandage, once a day for three weeks, following which it was observed that both warts had disappeared completely, leaving the skin flat, This patient was observed as never having a recurrence of the warts.

Apart from the foregoing examples, in vitro experimental data have been obtained which strongly indicate that the compositions of the present invention are highly active in inhibiting replication of the human papillomavirus. Tests were conducted to assess the activity of each of chloroquine, hydroxychloroquine and amodiaquine, as compared with the activity of cidofovir, a known inhibitor of replication of the human papillornavirus (“HPV”).

Specifically, in these tests, vectors for expression of an HPV genotype-matched set of viral E1 and E2 proteins (derived from virus strain HPV-11) along with an HPV ori-containing plasmid were cotransfected into HEK293 cells. The cells were cultured in the absence or presence of the test compounds (chloroquine, hydroxychloroquine, and amodiaquine), each at 1, 10, and 100 .mu.M concentrations. Low molecular weight DNA was harvested 2 days post-transfection and digested with DpnI and exonuclease III to remove unreplicated transfected plasmid DNA. The replicated DNA was then subjected to real time qPCR (quantitative polymerase chain reaction) analyses in triplicate. Two controls were performed. One was to omit the E1 expression vector to provide a background amount of undigested and unreplicated DNA. The other, positive control was treatment with the known inhibitor cidofovir, as mentioned above. A toxicity assay based upon cell viability at the time of harvest on day 2 was performed alongside each transient replication assay in 293 cells. The test compounds were added 4 hours post transfection for a total exposure of 44 hours. More than 1.times.10.sup.5 cells were scored in a BioRad Automatic Cell Counter, with a determination of the total numbers, the numbers of trypan blue stained (dead) cells, and the % of dead cells.

The results of these tests are set forth below in Table 1, in which EC.sub.50 represents the concentration of the tested compound that reduces viral replication by 50%, EC.sub.90 represents the concentration of the tested compound that reduces viral replication by 90%, CC.sub.50 represents the concentration of the tested compound that reduces cell viability by 50%, SI.sub.50 represents CC/EC.sub.50, and SI.sub.90 represents CC.sub.50/EC.sub.90

TABLE-US-00009 TABLE 1 Compound EC.sub.50 EC.sub.90 CC.sub.50 SI.sub.50 SI.sub.90 Cidofovir 148.00>200.00>200.00>1 1 Chloroquine 3.00 28.00>100.00>33>4 Hydroxychloroquine>100.00>100.00>100.00 1 1 Amodiaquine 5.00 58.00>100.00>20>2

Based on the experimental examples and test results summarized above, it is believed that the present invention comprises treatment methods and compositions that can treat infections caused by the human papillomavirus and can remove warts associated with that virus and prevent the recurrence of such warts, all by inhibiting replication of the virus. It is also believed that the removal of such warts, particularly the rectal and genital warts and those found in the oral cavity associated with the sexually transmitted types of the human papillomavirus, will reduce the risk of spreading that virus, and will therefore subsequently reduce the morbidity and mortality rates associated with cervical, vaginal, vulvar, penile, anal, rectal and/or oropharyngeal cancers. It is further believed that the methods and compositions of the present invention can be used as a prophylactic treatment, to provide protection during sexual intercourse from infections of the human papillomavirus for individuals who have not yet been infected.

It is also believed that the compositions and methods of the present invention may also be effective to treat infections in other mammalian species, caused by non-human papillomaviruses that may be specific to such species, and may be effective to remove warts associated with those viruses and to prevent the recurrence of such warts, all by inhibiting the replication of such non-human papillomaviruses in the same manner as with the human papillomavirus. This belief is based upon the close similarities among the various mammalian papillomaviruses, in terms of the organization of the viral genome, and in terms of the behavior of the viral proteins and the manner in which those proteins interact with host proteins. All papillomaviruses, regardless of type or the species they infect, need to modulate the host's immune system, which is accomplished with three viral proteins, designated ES, E6 and E7, which slightly modify the host's cells, making it difficult for the host's immune system to fight back.

Thus. since the target tissues, infection cycles and reproductive programs of all papillomaviruses are quite similar, independent of the papillomavirus type or the species infected, it is reasonable to conclude not only that all such viruses are dependent upon the same host properties and functions, but also that all such viruses would be susceptible to similar host inhibitors as well as pharmacological inhibitors, such as those which are disclosed herein. Accordingly, the applicant strongly believes that the compositions and methods of the present invention will be effective to treat infections of non-human papillomaviruses in other mammalian species, and to inhibit the replication of papillomaviruses in such species.

1. A method of topically treating an existing papillomavirus infection in a mammalian subject in need thereof, comprising administering topically to a localized area of infection of said mammalian subject a therapeutically effective amount of at least one compound selected from the group consisting of chloroquine, hydroxychloroquine, amodiaquine as the only active ingredient and respective pharmaceutically acceptable salts thereof, wherein said mammalian subject receives said topical administration once or twice a day for about two days to about two weeks.

2. The method of claim 1, wherein the pharmaceutical composition further comprises an antibiotic.

3. The method of claim 2, wherein the antibiotic is metronidazole.

4. The method of claim 1, wherein the pharmaceutical composition comprises chloroquine or a pharmaceutically acceptable salt thereof.

5. The method of claim 1, wherein the pharmaceutical composition comprises chloroquine or a pharmaceutically acceptable salt thereof, and metronidazole.

6. The method of claim 1, wherein the pharmaceutical composition comprises amodiaquine or a pharmaceutically acceptable salt thereof, and metronidazole.

7. The method of claim 1, wherein the pharmaceutical composition comprises chloroquine or a pharmaceutically acceptable salt thereof, amodiaquine or a pharmaceutically acceptable salt thereof, and metronidazole.

8. The method of claim 1, wherein said mammalian subject in need thereof has one or more epithelial lesions.

9. The method of claim 8, wherein said one or more epithelial lesions are selected from one or more of the group consisting of verrucae warts, flat warts, plantar warts, and anogenital warts of the skin and mucosal surfaces.

10. The method of claim 1, wherein said administering topically is providing said therapeutically effective amount of at least one compound in a form selected from the group consisting of a gel, a cream, a spray, a patch, a soap or other bathing apparatus, an ovule, a suppository, and aerosol droplets.

11. The method of claim 7, wherein the pharmaceutical composition is administered to the subject once per day.

12. The method of claim 7, wherein the pharmaceutical composition is administered to the subject twice per day.

13. The method of claim 7, wherein the pharmaceutical composition is administered to the subject for at least 1 week.

14. The method of claim 7, wherein the pharmaceutical composition is administered to the subject for at least 2 weeks.

15. The method of treatment of claim 8, wherein said epithelial lesions are verrucae warts.

16. The method of claim 8, wherein said epithelial lesions are flat warts.

17. The method of claim 8, wherein said epithelial lesions are plantar warts.

18. The method of claim 8, wherein said epithelial lesions are anogenital warts of the skin and mucosal surfaces.

19. The method of claim 1, wherein said mammalian subject in need thereof has virally-induced tumors that are associated with a papillomavirus.

20. A method of topically treating one or more virally-induced tumors that are associated with a human papillomavirus in a human in need thereof, comprising administering topically to a localized area of infection of said human a therapeutically effective amount of at least one compound selected from the group consisting of chloroquine, hydroxychloroquine, amodiaquine, and respective pharmaceutically acceptable salts thereof, wherein said human papillomavirus is selected from the group consisting of HPV-1, HPV-6, HPV-11, HPV-16, and HPV-18 and wherein said human receives said topical administration once or twice a day for about two days to about two weeks.

21. A method of topically treating an existing Human Papillomavirus (HPV) infection in a mammalian subject in need thereof, comprising administering topically to a localized area of infection on said mammalian subject a therapeutically effective amount of at least one compound selected from the group consisting of chloroquine, hydroxychloroquine, amodiaquine, and respective pharmaceutically acceptable salts thereof wherein said mammalian subject receives said topical administration once or twice a day for about two days to about two weeks.

22. The method of claim 21, wherein said therapeutically effective amount of at least one compound is administered together with at least one pharmaceutically acceptable non-active ingredient.

23. The method of claim 21, wherein said mammalian subject in need thereof has one or more epithelial lesions.

24. The method of claim 21, wherein said one or more epithelial lesions are selected from one or more of the group consisting of verrucae warts, flat warts, plantar warts, and anogenital warts of the skin and mucosal surfaces.

25. The method of claim 21, wherein said administering topically is providing said therapeutically effective amount of at least one compound in a form selected from the group consisting of a gel, a cream, a spray, a patch, a soap or other bathing apparatus, an ovule, a suppository, and aerosol droplets.

26. The method of claim 21, wherein said mammalian subject is a human.

27. The method of claim 25, wherein the pharmaceutical composition further comprises an antibiotic.

28. The method of claim 27, wherein the antibiotic is metronidazole.

29. The method of claim 21, wherein the pharmaceutical composition comprises chloroquine or a pharmaceutically acceptable salt thereof, amodiaquine or a pharmaceutically acceptable salt thereof, and metronidazole.
